(19) United States
(12) Patent Application Publication
(10) Pub. No.: US 2009/0222008 A1
(43) Pub. Date: Sep. 3, 2009

(54) REAMER AND METHOD OF REAMING

(76) Inventors: Alex Hogg, Leeds (GB); Michael Rock, Leeds (GB); John Dougall, Teeside (GB); Liam Rowley, Keighley (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/568,886

(22) PCT Filed: Mar. 24, 2005
(86) PCT No.: PCT/GB05/01173

§ 371 Date: Dec. 22, 2008

(30) Foreign Application Priority Data
Mar. 25, 2004 (GB) 0406821.9

(51) Int. Cl. A61B 17/00

(52) U.S. Cl. 606/80

(57) ABSTRACT

A collapsable reamer assembly and method of reaming a cavity into a bone are described. The reamer assembly comprises a base having a housing and an underside presented toward a surface of the bone in use. A collapsable reamer is rotatably mounted in the housing for rotation about a reaming axis directed away from the underside of the base. The collapsable reamer comprises at least one reaming component, supported for movement along the reaming axis from a collapsed state of the reamer to an extended state of the reamer to ream at least partially along the axis into the bone.

The present invention relates to a reamer and reaming method, and in particular to a reamer for use in reaming a cavity in a bone, and in particular the tibia.

There is a general move toward minimally invasive surgical procedures. Minimally invasive surgery has a number of considerations, including the size of incision used, which tends to have cosmetic consequences, and also the trauma suffered by body parts, such as soft tissues, ligaments, muscles, tendons and similar, effected by the surgical procedure at the surgical site.

For example, during knee surgery, and by way of a particular example total knee replacement surgery, it can be required to attach an implant or prosthesis to the proximal end of the tibia where it interfaces with the femur. In order to be mounted securely on the tibia, the implant can include a stem which is located within a cavity formed along the longitudinal axis of the tibia. Therefore as part of this procedure, it can be necessary to drill or ream a cavity of the correct size and depth in the tibia to receive a stem of the implant.

In one surgical procedure, the femur is subluxed to a level sufficient to expose the entire top surface of the tibia. The top surface is then resected to a flat surface. A plate is mounted on the surface and a large tower is mounted on the plate and extends substantially perpendicularly away from the surface to receive and guide a reamer, to form a cavity in the tibia.

However, in order to carry out such a method of forming a cavity a large incision is required in order to locate the large tower on the resected tibia surface. This method also requires the femur to be subluxed to a level sufficient to allow access of the reamer along the axis of the tibia which can result in damage to the tendons and other soft body parts adjacent to the knee.

Hence the above method and reaming apparatus may not be suitable for use in a minimally invasive procedure as large incisions may be required and soft body parts at the surgical site can be damaged. Therefore, the present invention addresses problems associated with reaming a cavity as part of a minimally invasive surgical technique.

According to a first aspect of the invention, there is provided a reamer assembly for reaming a cavity substantially along an axis into a bone. The assembly comprises a base, having an underside and a housing, and a collapsable reamer. The collapsable reamer is rotatably mounted in the housing for rotation about a reaming axis directed away from the underside.

As the reamer is collapsable, the reaming assembly has a small form. Hence, the reamer can be used in confined spaces and therefore helps to minimise the surgical trauma that may otherwise be experienced by soft tissues at a surgical site. Also, a smaller incision can be used to introduce the reaming assembly to the surgical site.

The collapsable reamer can comprise at least one reaming component, supported for movement along the reaming axis from a collapsed state of the reamer to an extended state of the reamer to ream at least partially along the axis of the bone.

The collapsible reamer can comprise at least two concentric reaming components, supported for relative movement along the reaming axis.

The term concentric is used herein in its general sense to refer to components which are aligned along a common central axis. Hence, concentric encompasses both collapsed and extended reaming component configurations in which the reaming components are coaxial, with the reaming axis forming the common axis and the reaming components extending and collapsing along the reaming axis.

Preferably, in the collapsed state, the length of the reamer assembly along the reaming axis is substantially equal to or less than the largest dimension of the base along the reaming axis. This ensures that the length of the reamer assembly is minimised, enabling the use of a smaller incision.

Preferably, the housing defines an aperture through the base. The reaming components can then be located within this aperture giving a simple construction and simplifying the assembly of the reamer. This also facilitates connection of a rotational drive as direct access to the collapsable reamer is provided.

Preferably, the reaming component and housing, or the reaming components, overlap in the direction along the reaming axis. The overlap can provide a retaining mechanism which ensures that the reaming component and housing or reaming components do not disengage as they move between the collapsed and extended configurations.

Each reaming component can have a substantially right cylindrical shape. The cross section of the cylinder can be curved, for example circular, oval or elliptical, or polygonal, for example triangular, square, pentagonal, and similar.

Preferably, each reaming component has a generally tapered shape. An outer surface of each reaming component can provide a cutting formation. In the collapsed state, at least one reaming component can be enclosed on its tapered side by another reaming component or by the base.

The generally tapered shape of the reaming component allows a tapered cavity to be formed. The surface of the reaming components provides a cutting formation, which simplifies the number of components required in the reaming assembly. Furthermore, because at least one reaming component is enclosed on its tapered sides by another reaming component or by the base in the collapsed state, this ensures that the reaming component is only in contact with a surface to be reamed when it is at least partially extended from the collapsed configuration.

In one embodiment, at least a part or the whole of each reaming component has a generally conical or frusto-conical shape. This allows the reaming components to ream evenly around the tapered surface of the cone simultaneously. Each reaming component can have a cylindrical body part and a conical or frusto-conical reaming part.

In another embodiment, each reaming component has a generally trapezoidal shape. The reaming components can therefore be moved relative to each other more easily, as the area in contact between them, and therefore the frictional forces, is reduced. Each reaming component can have a generally triangular shape. In the case of a reamer with more than one reaming component, this construction can also avoid the formation of “steps” in the cavity wall caused by the interface between the individual reaming components. Preferably, the reaming components are shaped such that, in the extended state, they provide a substantially smooth and/or continuous cutting surface so as to provide a substantially smooth cavity wall.

Preferably, the at least two reaming components have a slotted construction for relative movement along the reaming axis. This allows the reaming components to move relative to each other with a simple construction.

Preferably, the at least two reaming components are restrained against relative rotational movement. Thus, when the collapsable reamer is extended, all of its parts rotate in unison.

Preferably, the at least two reaming components are restrained against relative rotational movement by a keyed engagement between mutually adjacent sides of the reaming components. The keyed engagement can be provided by respective formations on adjacent surfaces of reaming components. A key engagement is a simple mechanism to ensure that the components are locked relative to each other. This engagement can also be combined with the slotted construction which allows relative movement along the reaming axis to further simplify the construction.

Preferably, the assembly further comprises mounting holes formed in the base for securing the reamer assembly to the bone surface in use. This ensures that the reaming assembly does not move during reaming, allowing the cavity to be reamed to an accurate size or fixed position. The mounting holes can be disposed on generally opposite sides of the housing.

Preferably, the reaming assembly further comprises a generally circular edge within the housing and a corresponding generally circular formation formed on mutually adjacent sides of the reaming components, such as a lip. This allows the housing the retain the reamer component in the extended state. This ensures that the cavity cannot be formed deeper than desired.

According to a further aspect of the invention there is provided a method of reaming a cavity substantially along an axis into a bone using a reamer assembly having a collapsable reamer extensible along a reaming axis from a collapsed state of the reamer to an extended state of the reamer. The method can comprise locating the reamer assembly on a surface of the bone, imparting rotational drive to the collapsable reamer, and reaming a cavity by extending the collapsable reamer from the collapsed state toward the extended state along the reaming axis.

This method allows a cavity to be reamed using a collapsable reamer. The collapsable reamer can be used in smaller surgical sites than conventional reamers and can be inserted through a relatively small incision. Hence, an improved minimally invasive surgical procedure is provided.

The method can include resecting a surface of the bone before locating the reamer on the resected surface of the bone.

Preferably, the method further comprises the step of securing the reamer assembly to the surface. This ensures that the reamer assembly cannot move during reaming and that the cavity is reamed to an accurate size and/or fixed position.

A rotational drive system can be provided colinear to the reaming axis. This allows a simple drive system to be realised.

Preferably, a rotational drive system can be provided wholly or at least partially at an acute angle to the reaming axis. This further facilitates use of the reamer assembly at confined surgical sites.

Preferably, the rotational drive system engages a reaming component which is distal most when the reamer is in the extended state. Thus, only a single reamer component is driven directly by the rotational drive which allows easier connection and simplicity in use.

The bone can be a tibia. The method can be used as part of an orthopedic implant surgical procedure and more particularly as part of a tibial preparation procedure.

Embodiments of the invention will now be described, by way of example and not in any limitative sense, with reference to the following figures in which:

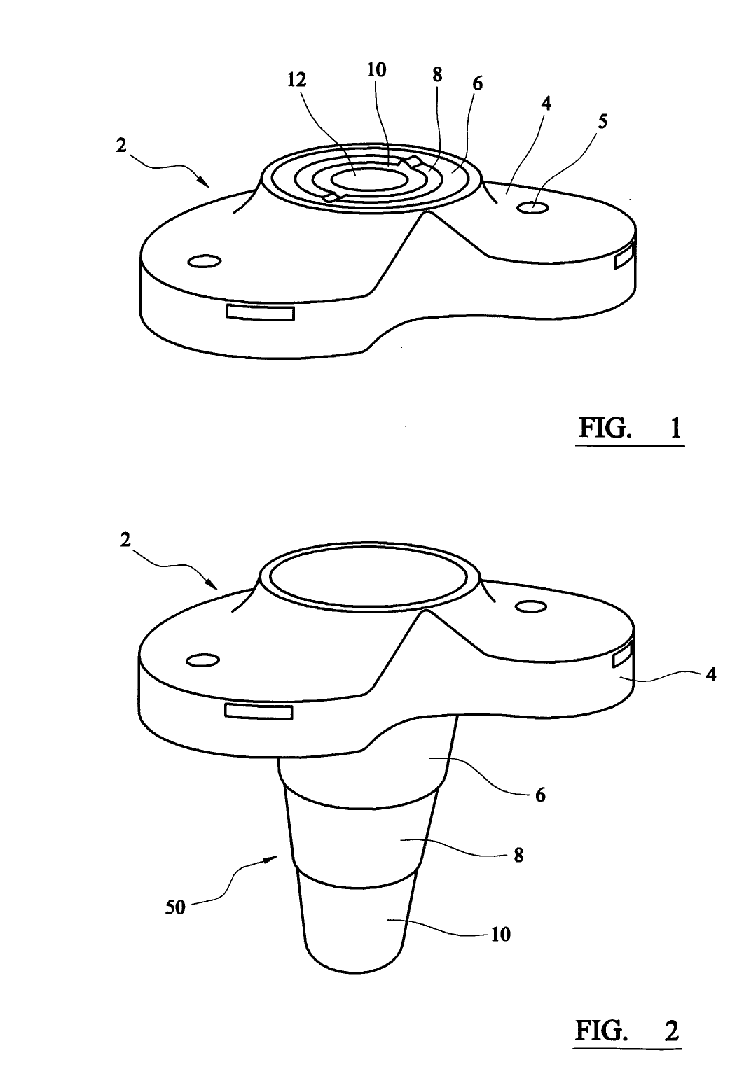

FIG. 1 is a schematic perspective view of a first embodiment of the present invention in a collapsed state;

FIG. 2 is a perspective view of the first embodiment of the invention as shown in FIG. 1 in an extended state;

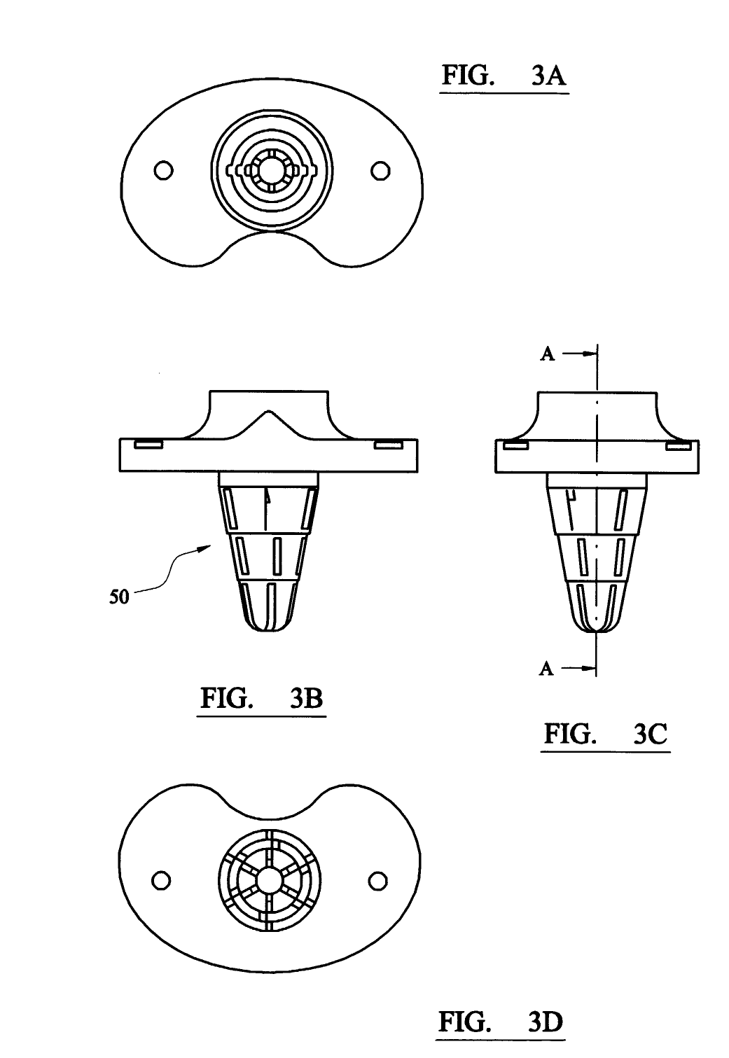

FIGS. 3A-3D show top, front, bottom and side elevations respectively of the first embodiment in greater detail;

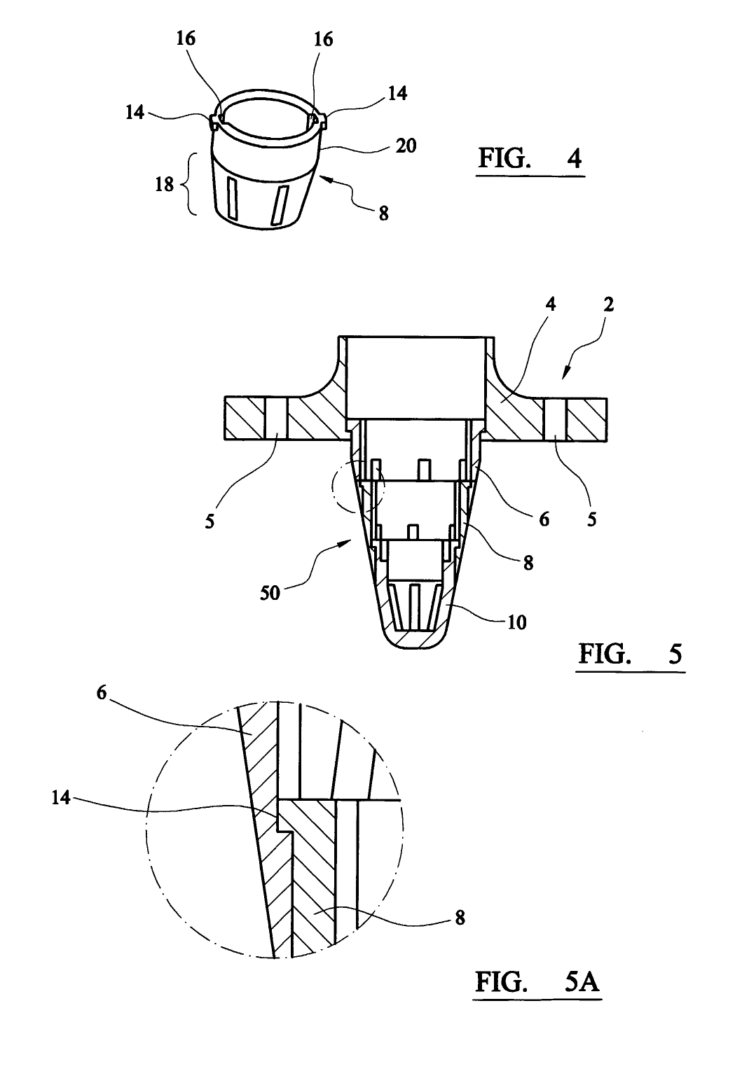

FIG. 4 is a perspective view of a reaming component of the first embodiment;

FIG. 5 is a cross section along line AA′ of FIG. 3D;

FIG. 5A shows an expanded view of a part of FIG. 5 in greater detail;

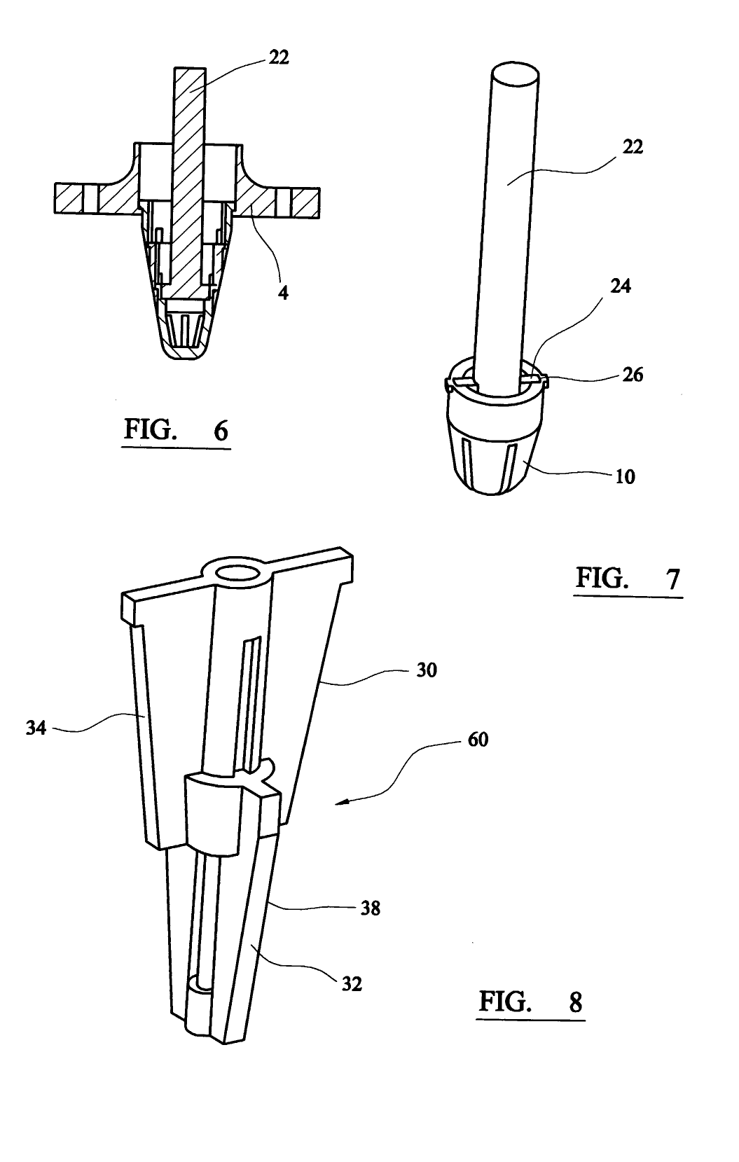

FIG. 6 is a cross section similar to that shown in FIG. 5 with the first embodiment in the extended state and showing the connection of a drive shaft;

FIG. 7 is an isometric view of the drive shaft connected to a distal most reaming component; and

FIG. 8 shows a schematic perspective view of reaming components of a second embodiment of the present invention.

Like items appearing in different figures share common reference numerals unless indicated otherwise.

The invention will be described with particular reference to the example of reaming a cavity in a tibia as part of a tibial preparation procedure in a total knee replacement operation. However, the reamer assembly and method of reaming are not limited to that specific use and can be used to ream a cavity in any bone and as part of any surgical procedure in which a cavity is reamed in a bone.

FIG. 1 shows a schematic depiction of a reaming assembly 2 according to a first embodiment of the present invention. The reaming assembly 2 is shown in FIG. 1 with a collapsable reamer in a collapsed state.

The reaming assembly 2 comprises a base 4 which is approximately the same size as the resected surface of a bone, e.g. the top end of a tibia, in which a cavity is to be formed. The base 4 includes a housing having an aperture in which three reaming components 6, 8, 10 are concentrically and rotatably mounted. In the collapsed state, as shown in FIG. 1, the reaming components 6, 8, 10 are all enclosed within the depth of the base 4.

The reaming components fit within each other, in a telescopic manner, and are centred on a central axis of the aperture.

The base 4 also includes first and second mounting holes 5 disposed on opposite sides of the aperture. The mounting holes can be used to receive fastenings, such as a pin or screw, to secure the base to a resected bone surface. This ensures that the base does not move during the reaming operation, allowing the cavity to be formed accurately.

FIG. 2 shows the reaming assembly 2 with the collapsable reamer 50 in an expanded state. It can be seen that the reaming component 10 which is innermost within the aperture when in the collapsed state forms the distal end of the reamer in the extended state. The interior of the distal most reaming component 10 incorporates a fitting 12, in the form of a pair of diametrically opposed slots 26, for receiving mating lugs 24 of a rotational drive.

FIGS. 3A-3D show top, front, bottom and side views respectively of the reaming assembly shown schematically in FIGS. 1 and 2 in greater detail. FIG. 4 depicts the configuration of the reaming component 8 in isolation from the other parts of the collapsable reamer 50. Reaming components 6, 8 and 10 have a generally right circular symmetric upper body part and a generally frusto-conical shaped lower reaming part.

As also illustrated in FIG. 4, reaming component 8 incorporates tabs 14 which engage with a corresponding slot in the outer surface of adjacent reaming component 6, within which reaming component 8 is nested. An inner surface of the reaming component also incorporates a slot 16 which receives a corresponding tab from the outer surface of adjacent reaming component 10, which is nested within reaming component 8. Through the interaction of the slots 16 and tabs 14 the reaming components are able to move axially relative to one another, and yet are also locked for rotational movement. This means that when one of the reaming components is rotated, the other of the reaming components also rotate due to the keyed engagement of the slots and tabs. Hence the slots and tabs provide a mechanism allowing relative translation of reaming components along the reaming axis while rotating in unison.

As illustrated in FIG. 4 each reaming component includes cutting formations or features on a conical, tapered outer surface of a reaming part 18 of the reaming component 8. Other arrangements of cutting features could be used with similar effect. Above the conical section 18 of the reaming component 8, there is a generally cylindrical body section 20. In the extended configuration, the cylindrical section 20 is retained within the outwardly adjacent reaming component 6.

FIG. 5 shows a cross section through the reaming assembly 2 with the collapsable reamer 50 in the extended configuration. The detail shown in FIG. 5A shows how the tab 14 in the reaming component 8 interacts with a shelf towards the distal end of the outwardly adjacent reaming component 6 to ensure that the reaming component 8 cannot escape from overlapping contact with the reaming component 6. This provides a limit to the extension of the reaming component and defines the maximum depth to which the reaming components can be extended from the base 4. It can be seen that a similar tab and edge formation is found between the distal most reaming component 10 and the reaming component 8, and also between the proximal most reaming component 6 and a peripheral portion of the housing in the base 4. This latter feature prevents the reamer from escaping from the base in use.

FIG. 6 shows a cross section through the reaming assembly 2 illustrating the connection of a drive shaft 22 to impart rotational drive, or torque, to the collapsable reamer 50. A pair of lugs 24 on the free end of drive shaft 22 engage with slots 12 contained in the distal most reaming component 10. FIG. 7 shows an isometric view of the drive shaft 22 mounted in the distal most components 10. It can be seen that the drive shaft engages with the reaming component through the projections 24 which interface with slots 26 formed in the drive connector 12 of the distal most reaming component 10.

This construction allows the drive shaft 22 to have a smaller diameter than the distal most reaming component 10. This means the drive shaft is considerably smaller in diameter than the maximum diameter of the cavity formed. The access required for the drive shaft is correspondingly reduced.

The drive shaft can have a universal joint, or some other form of coupling, which allows drive to be supplied at an acute angle to the reaming axis. Hence, in this way access to the reaming assembly only along the reaming axis is not required. In alternative embodiments, drive can be supplied substantially perpendicularly to the reaming axis. For example gearing can be used to rotate the rotational drive through ninety degrees. For example an input drive shaft can drive a worm gear which meshes with a gear cog provided by teeth around the outer periphery of the outermost reaming component 6, so as to transmit drive through ninety degrees to the collapsable reamer. A thread can also be provided to drive the other reaming components down.

An example method of forming a cavity in a bone using the reaming assembly of the present invention will now be described. Various conventional steps may be carried out preceding and following the operations to be described, as will be appreciated by those of ordinary skill in the art, but they have not been described herein so as not to obscure the nature of the present invention. The surgeon first makes an incision and then resects a surface of the bone to a flat surface to receive the underside of the base of the reaming assembly. The reaming assembly is then placed on the resected surface and secured with pins. A drive mechanism is then attached by engaging the free end to the connector provided in the interior of the distal most reaming component. The drive is then activated and the distal most reaming component rotates and can be urged toward the bone and translates along the reaming axis relative to its adjacent reaming components to begin reaming the cavity. After a certain depth of reaming, corresponding to a certain extension of the collapsable reamer, the distal most reaming component has moved as far along the reaming axis relative to the next reaming component that the tab engages with the corresponding shelf on the next reaming component. From this point on the distal most and the next reaming component continue to extend downward by relative movement of the next reaming component with respect to its outwardly adjacent reaming component. Thus, the reaming assembly extends downwards along the reaming axis to form a cavity.

Once the reaming components are extended fully, the cavity has been formed to the correct depth and the drive can be reversed to retract the reamer assembly to the collapsed state. In an alternative embodiment the drive is removed from the cavity, pulling the reaming components with it.

The drive mechanism can then be detached and the collapsed reaming assembly removed via the original incision.

A second embodiment of the present invention is depicted in FIG. 8. FIG. 8 shows only the collapsable reamer 60 component, the base plate and drive shaft are omitted. The base plate used for this collapsable reamer is similar to that described previously. This embodiment includes two reaming components 30 and 32. The reaming components are generally trapezoidal in shape and have a slotted construction which allows the two components to slide relative to one another along a reaming axis and yet stay rotationally rigid. The reaming surfaces in this embodiment are formed by the non parallel sides of the trapezoidal section, surfaces 34 and 38 and their diametrically opposite surfaces as depicted in FIG. 8.

In alternative embodiments of the reaming assembly the tapering sections of the reaming component has a shape other than conical, frusto-conical or trapezoidal. For example, the reaming component could be hexagonal, or octagonal or generally any other tapered shape.

In other alternative embodiments, the number of tapered sections varies depending on the depth and profile of the cavity to be reamed. In particular, the depth of the collapsed state can be reduced by increasing the number of reaming components. The tapers on each reaming component and length in the reaming direction of the reaming surface can also be adjusted to allow the assembly to ream the profile of cavity required. In particular, as well as the two or three section described above in relation to the embodiments, it would be appreciated that other numbers of sections are also possible, for example 4, 5, 6 and so on.

In other embodiments, the keyed engagement by which the reaming components are connected to transfer rotational drive is only made when a component is fully extended, this ensures that the rotational drive only drives both components which are currently active in reaming a cavity, maximising the efficiency of the drive.

In another alternate embodiment, the distal most reaming section includes a self tapping screw thread at its distal end. The action of this self tapping screw acts to draw the distal most reaming component into the bone and eliminates the need for the drive to provide a pressure toward the bone. This provides more flexibility in positioning the drive.

In a further alternative embodiment, only a single reaming component is provided, resulting in a simpler construction of the reaming assembly.

The method of use of the reaming assembly is particularly suited to use in reaming a cavity in a tibia for an implant as part of a knee replacement surgical procedure. However, the method can be used in other surgical procedures in which it is required to ream a cavity in a bone. Also, some of the method steps described are either optional or can be modified. For example if a bone surface is sufficiently flat, or if the underside of the base is appropriately shaped, no resecting step may be required. Also, the drive or a part of the drive may already be connected to the reaming assembly before securing to the bone. Therefore modifications and variations in the specific method described are envisaged. Also, it will be appreciated that features of a one of the embodiments can be mixed and matched with features of the other embodiments.

1. A reamer assembly for reaming a cavity substantially along an axis into a bone, the reamer comprising:
a base having a housing and an underside presented toward a surface of the bone in use; and
a collapsable reamer rotatably mounted in the housing for rotation about a reaming axis directed away from the underside of the base, the collapsable reamer comprising at least one reaming component, supported for movement along the reaming axis from a collapsed state of the reamer to an extended state of the reamer to ream at least partially along the axis into the bone.

2. The reaming assembly of claim 1, wherein in the collapsed state, the length of the collapsable reamer along the reaming axis is substantially equal to or less than the largest dimension of the base along the reaming axis

3. The reaming assembly of claim 1, wherein the housing defines an aperture through the base.

4. The reaming assembly of claim 1, wherein the or each reaming component has a generally tapered shape, an outer surface of the or each reaming component provides a cutting formation, and wherein, in the collapsed state, the or each reaming component is enclosed on its tapered sides another reaming component or by the base.

5. The reaming assembly of any preceding claim 1, wherein the or each reaming component has a part with a generally conical or frusto-conical shape.

6. The reaming assembly of any preceding claim 1 wherein the or each reaming component has a generally trapezoidal or triangular shape.

7. The reaming assembly of any preceding claim 1, further comprising mounting holes formed in the base for receiving a fastening to secure the reamer assembly to the bone surface in use.

8. The reaming assembly of any preceding claim 1, further comprising a generally circular edge within the housing and a corresponding generally circular lip formation formed on mutually adjacent sides of at least one reaming component.

9. The reaming assembly of any preceding claim 1, and further comprising at least two concentric reaming components, supported for relative movement along the reaming axis.

10. The reaming assembly of claim 9, wherein the at least two reaming components overlap in the direction along the reaming axis.

11. The reaming assembly of claim 9, wherein the at least two reaming components have a slotted construction for relative movement along the reaming axis.

12. The reaming assembly of any of claims 9, wherein the at least two reaming components are restrained against relative rotational movement.

13. The reaming assembly of claim 12, wherein the at least two reaming components are restrained against relative rotational movement by a keyed engagement between formations provided on mutually adjacent sides of the reaming components.

14. A method of reaming a cavity substantially along an axis into a bone using a reamer assembly having a base with a housing and a collapsable reamer rotatably mounted in the housing for rotation about a reaming axis directed away from the base, the collapsable reamer being extensible along the reaming axis from a collapsed state of the reamer to an extended state of the reamer, the method comprising:
locating the reamer assembly on a surface of the bone with the reaming axis in registration with the axis;
imparting rotational drive to the collapsable reamer; and
reaming a cavity into the bone by extending the collapsable reamer from the collapsed state toward the extended state along the reaming axis.

15. The method of claim 14, and further comprising resecting the surface of the bone before locating the reamer assembly on the resected surface.

16. The method of claim 14, and further comprising securing the reamer assembly to the surface of the bone.

17. The method of any of claims 14, wherein a rotational drive system is attached to the reaming assembly co-linearly with the reaming axis.

18. The method of any of claims 14, wherein a rotational drive system is attached to the reaming assembly at least partially at an acute angle to the reaming axis.

19. The method of any of claims 14, wherein rotational drive is supplied to the reaming assembly along a direction substantially perpendicular to the reaming axis.

20. The method of any of claims 17, wherein the rotational drive system is attached to a reaming component which is the distal most reaming component when the reamer is in the extended state.
